(19) United States
(12) Patent Application Publication
(10) Pub. No.: US 2025/0144176 A1
(43) Pub. Date: May 8, 2025

(54) COMPOSITIONS COMPRISING OSTEOPONTIN (OPN)

(71) Applicant: SOCIETE DES PRODUITS NESTLE S.A., Vevey (CH)

(72) Inventors: ELIZABETH FORBES-BLOM, La Conversion (CH); MARIO NOTI, Vulliens (CH); CHEONG KWET CHOY KWONG CHUNG, Lausanne (CH)

(21) Appl. No.: 18/834,297

(22) PCT Filed: Jan. 31, 2023
(86) PCT No.: PCT/EP2023/052311

§ 371 Date: Jul. 30, 2024

(30) Foreign Application Priority Data
Jan. 31, 2022 (EP) 22154312.7

(51) Int. Cl. A61K 38/19; A23L 33/00; A23L 33/135; A23L 33/18; A23L 33/21; A61K 31/702; A61K 35/741; A61P 11/00; A61P 31/14; A61P 37/02

(52) U.S. Cl. CPC A61K 38/19; A23L 33/135; A23L 33/18; A23L 33/21; A23L 33/40; A61K 31/702; A61K 35/741; A61P 11/00; A61P 31/14; A61P 37/02

(57) ABSTRACT

The present invention relates to compositions comprising osteopontin (OPN) for the treatment and/or prevention of viral infections in mammals, such as humans, in particular infants, young children and pre-schoolers.

### FIELD OF THE INVENTION

The present invention relates to compositions comprising osteopontin (OPN) for the treatment and/or prevention of viral infections in mammals, such as humans, in particular infants, young children and pre-schoolers.

### BACKGROUND OF THE INVENTION

A viral infection occurs when the virus proliferates inside the host's cells and hence utilises the host's resources to promote its own multiplication. Viral infections can also interfere with the normal functioning of the host and may lead to more severe infection-related disorders, including increased risk of concurrent or secondary bacterial infections and associated antibiotic use as well as long term alterations and subsequent inflammatory diseases later in life.

Viral respiratory infections, such as respiratory syncytial virus (RSV), affect nearly 90% of children by the age of two (Karpinnen et al, Clin Microbiol Infect, 2016; 22; 208.e1-e6) and often lead to bronchiolitis, an inflammatory bronchial reaction in infants and young children (Pickles et al, J Pathol, 2015; 235; 266-276). Respiratory viruses primarily infect airway epithelium. Higher viral loads have been associated with increased bronchiolitis severity and conversely, rapid viral load reduction in infants was associated with faster disease resolution (Pickles et al, J Pathol, 2015; 235; 266-276). It is well documented that infected and necrotic epithelial cells contribute to the airway obstruction and inflammation during RSV infection (Pickles et al, J Pathol, 2015; 235; 266-276) and as such epithelial cell sloughing is a feature of viral bronchiolitis and associated with disease severity (Johnson et al, Mod Pathol, 2007; 20; 108-119). Plasmacytoid dendritic cells (pDC) are known to be protective against pathology during RSV infection, and adaptive immune responses including CD4+ and CD8+ T cells are important in viral elimination from the respiratory tract (Openshaw et al, Annu Rev Immunol, 2017; 35; 501-532). However, if the immune defense response is dysregulated, inflammatory granulocytes such as neutrophils along with CD4+ and CD8+ T cell responses can also lead to immunepathology following respiratory viral infection (Newton et al, Semin Immunopathol, 2016; 38; 471-482). Moreover, such uncontrolled inflammatory responses can also lead to pathological airway smooth muscle remodeling, a hallmark feature of asthma reported to commence in early life (O'Reilly et al, JACI, 2013; 131; 1024-1032) as well as playing a central role in the pathogenesis of chronic obstructive pulmonary disease (COPD) (Yan F et al, J Transl Med, 2018; 16; 262-270).

Breastfeeding is a recognised factor that reduces the severity of viral infection in infants.

It has previously been demonstrated that dietary OPN delivered beneficial effects within the gut and systemically, including protective immune responses. Early life dietary supplementation with bovine OPN reduced inflammation resulting from lipopolysaccharide (LPS) administration (Jiang et al. JPGN, 2020; 71; 125-131) and infants fed formula supplemented with bovine OPN had an immune cell profile more similar to breastfed infants (West et al. Pediatr Res, 2017; 82; 63-71). In addition, oral administration of mammalian milk OPN enhanced immune resistance to an infectious disease following administration of a vaccine via strengthened humoral immunity (WO 2015/001092 A1).

### SUMMARY OF THE INVENTION

Using a neonatal mouse model of pneumonia virus of mice (PVM)-induced bronchiolitis and early life nutritional interventions, the present inventors surprisingly demonstrated that a lack of dietary OPN increases susceptibility to respiratory viral infection. Pups nursed by OPN-deficient dams exhibited an increase in viral burden, dysregulated immune responses and pathological airway smooth muscle remodeling compared to controls reared by OPN-sufficient dams. Dietary supplementation with OPN was sufficient to reduce viral load, airway epithelial sloughing and pathological airway smooth muscle remodeling. Specifically, early life supplementation with OPN generated an enhanced immune defense characterized by dynamic increases in lung plasmacytoid dendritic cells (pDC), CD4⁺ and CD8⁺ T cell responses at the peak of infection that was associated with a rapid resolution of inflammation upon viral clearance.

These findings support a critical function of OPN in providing protection against viral infections, in particular viral bronchiolitis, in early life and uncover functional benefits of OPN to mount effective anti-viral immune responses associated with faster disease resolution. Given that viral airway infections in early life represent a major independent risk factor for subsequent asthma, dietary OPN supplementation may also prevent long-term complications associated with respiratory tract infections in early life.

Accordingly, in a first aspect the present invention relates to a composition comprising osteopontin (OPN) for use in the treatment and/or prevention of a viral infection, in particular a respiratory viral infection, in a mammal.

The first aspect of the invention may also be worded as a method for treating and/or preventing a viral infection, in particular a respiratory viral infection, in a mammal, said method comprising administering a composition comprising osteopontin (OPN) to said mammal.

Alternatively, the first aspect of the invention may also be worded as the use of osteopontin (OPN) for the manufacture of a composition for the treatment and/or prevention of a viral infection, in particular a respiratory viral infection, in a mammal.

In a second aspect, the present invention concerns a composition comprising osteopontin (OPN) for use in reducing the risk of contracting a viral infection, in particular a respiratory viral infection, in a mammal and/or for use in reducing the symptoms associated with a viral infection, in particular a respiratory viral infection, in a mammal.

The second aspect of the invention may also be formulated as a method for reducing the risk of contracting a viral infection, in particular a respiratory viral infection, in a mammal and/or for reducing the symptoms associated with a viral infection, in particular a respiratory viral infection, in a mammal, said method comprising administering a composition comprising osteopontin (OPN) to said mammal.

Alternatively, the second aspect of the invention may also be worded as the use of osteopontin (OPN) for the manufacture of a composition for reducing the risk of contracting a viral infection, in particular a respiratory viral infection, in a mammal and/or for reducing the symptoms associated with a viral infection, in particular a respiratory viral infection, in a mammal.

In a third aspect, the present invention concerns a composition comprising osteopontin (OPN) for use in preventing or reducing the risk of contracting a bacterial co-infection and/or a bacterial secondary infection associated with a viral infection, in particular a respiratory viral infection, in a mammal.

Alternatively worded, the third aspect concerns a method for preventing or reducing the risk of contracting a bacterial co-infection and/or a bacterial secondary infection associated with a viral infection, in particular a respiratory viral infection, said method comprising administering a composition comprising osteopontin (OPN) to said mammal.

The third aspect may also be worded as the use of osteopontin (OPN) for the manufacture of a composition for preventing or reducing the risk of contracting a bacterial co-infection and/or a bacterial secondary infection associated with a viral infection, in particular a respiratory viral, in a mammal.

In a fourth aspect, the present invention is directed to a composition comprising osteopontin (OPN) for use in preventing or reducing the risk of allergen sensitisation and/or developing an allergic respiratory tract disease in a mammal.

The fourth aspect of the invention may, alternatively, be expressed as a method for preventing or reducing the risk of allergen sensitisation and/or developing an allergic respiratory tract disease in a mammal, said method comprising administering a composition comprising osteopontin (OPN) to said mammal.

A further alternative formulation of the fourth aspect of the invention is the use of osteopontin (OPN) for the manufacture of a composition for preventing or reducing the risk of allergen sensitisation and/or developing an allergic respiratory tract disease in a mammal.

Finally, in a fifth aspect, the present invention relates to a composition comprising osteopontin (OPN) for use in preventing or reducing the risk of pulmonary diseases, including chronic obstructive pulmonary disease, in a mammal.

Alternatively worded, the fifth aspect concerns a method for preventing or reducing the risk of pulmonary diseases, including chronic obstructive pulmonary disease, said method comprising administering a composition comprising osteopontin (OPN) to said mammal.

A further alternative formulation of the fifth aspect of the invention is the use of osteopontin (OPN) for the manufacture of a composition for preventing or reducing the risk of pulmonary diseases, including chronic obstructive pulmonary disease, in a mammal.

### DESCRIPTION OF THE FIGURES

FIG. 1 shows a schematic outline of the neonatal mouse model of pneumonia virus of mice (PVM)-induced bronchiolitis.

FIG. 2 shows that OPN-supplementation early in life of pups receiving OPN-deficient mother's milk reduces viral load and sloughing in the airways. (a) Quantification of PVM⁺ airway epithelial cells (AECs) expressed as percentage of infected AECs at 5 dpi. (b) Extent of epithelial sloughing in the airways at 9 dpi.

FIG. 3 shows that OPN-supplementation early in life of pups receiving OPN-deficient mother's milk promotes increased number of protective plasmacytoid dendritic cells (pDCs) in the airways of virus-infected mice.

FIG. 4 shows that OPN-supplementation early in life of pups receiving OPN-deficient mother's milk promotes an appropriate CD4⁺ cell response to airway viral infection. (a) Temporal regulation of CD4⁺ cell numbers in lung tissues at 5 dpi (peak of viral infection) and (b) 9 dpi (peak of immunopathology) of indicated groups.

FIG. 5 shows that OPN-supplementation early in life of pups receiving OPN-deficient mother's milk promotes appropriate immune defence and control (resolution) of the CD8⁺ cell in response to airway viral infection. (a) Temporal regulation of CD8⁺ cell numbers in lung tissues at 5 dpi (peak of viral infection) and (b) 9 dpi (peak of immunopathology) of indicated groups.

FIG. 6 shows that OPN-supplementation early in life of pups receiving OPN-deficient mother's milk reduces the number of neutrophils in lung tissue at the peak of immunopathology (9 dpi).

FIG. 7 shows that OPN-supplementation early in life of pups receiving OPN-deficient mother's milk attenuates pathological airway smooth muscle remodeling. FIG. 7 shows quantification of airway smooth muscle (ASM) area in lung tissues of the indicated groups at 9 dpi.

### DETAILED DESCRIPTION OF THE INVENTION

### Definitions

Osteopontin is abbreviated “OPN”.

“Probiotic” means microbial cell preparations or components of microbial cells with a beneficial effect on the health or well-being of the host. (Salminen et al, Trends Food Sci Technol, 1999; 10; 107-10). The term “probiotic” includes viable as well as non-viable (killed) microorganisms.

“Prebiotic” means a food substance that promotes the growth of the gastrointestinal microflora. Prebiotics are not broken down in the stomach and/or upper intestine or absorbed in the gastrointestinal tract of the person ingesting them, but they are fermented by the gastrointestinal microflora and/or by probiotics, cf. Gibson et al, J Nutr, 1995; 125; 1401-1412.

Bifidobacterium longum subspecies infantis is referred to herein as “Bifidobacterium infantis”.

In the present context the term “infant” means a child having an age of 12 months or younger.

In the present context, the terms “young child” or “toddler” are used interchangeably and refer to a child having an age between 12 and 36 months.

Herein, the term “pre-schoolar” refers to a child having an age from 3 to 5 years of age.

The expression “nutritional composition” means a composition that nourishes a mammal, preferably a human, and may comprise a lipid (fat) source, a protein source and a digestible carbohydrate source. A “nutritional composition”, as used herein, is not human breast milk. Non-limiting examples of a nutritional composition are infant formula, growing-up milk, baby food, infant cereals, fortifiers and supplements.

The term “WPC” is an abbreviation of “whey protein concentrate”.

“Composition”, as used herein, is not human breast milk.

The expression “infant formula” means a nutritional product intended for nutritional use by infants during their first 12 months of life, and which satisfies the nutritional requirements of said infants. For further details on infant formulas reference is made to the Commission Directive 2006/141/EC of 22 Dec. 2006 on infant formulae and follow-on formulae. The expression “infant formula” encompasses both a “starter infant formula”, i.e. a foodstuff particularly intended for nutritional use by infants during their first 4-6 months of life, and a “follow-on formula”, i.e. a foodstuff particularly intended for nutritional use by infants aged over 4-6 months.

The expressions “growing-up milk” or “toddlers' milk” are nutritional products adapted specifically to young children (toddlers).

The terms “baby food” and “infant cereal compositions” refer to foodstuff intended for nutritional use by infants during their first 12 months of life.

The term “fortifier” refers to liquid or solid nutritional compositions suitable for mixing with human milk or infant formula. The fortifier may be packaged in single doses.

The term “supplement” means a foodstuff containing specific nutrients and/or other beneficial ingredients intended to supplement a diet. The “supplement” may be in the form of a powder or an oil and may be added to the diet as is, or it may be combined with other ingredients, such as maltodextrin, before being added to the diet. A “supplement” may also be added to expressed human breast milk.

The terms “respiratory tract infection” and “airway infection” are used synonymously herein.

In the present context, the term “gastrointestinal tract” includes the mouth, pharynx, oesophagus, stomach, small intestine, large intestine, rectum and anus. The term “intestine” includes the small intestine, the large intestine and rectum.

The human milk oligosaccharide “difucosyllactose” (abbreviated “DFL”) is synonymous with “lacto-difucotetraose” (abbreviated “LDFT”).

Herein, “LA” refers to linoleic acid, and “ALA” refers to α-linolenic acid.

“PA” refers to palmitic acid (C16:0).

“LC-PUFA” refers to long chain polyunsaturated fatty acids having at least 20 carbon atoms in the fatty acyl chain and 2 or more unsaturated bonds. “DHA” refers to docosahexaenoic acid (22:6, n3); “EPA” refers to eicosapentaenoic acid (20:5 n3); “ARA” refers to arachidonic acid (20:4 n6); “DPA” refers to docosapentaenoic acid (22:5 n3).

The term “and/or” when used in the context of “X and/or Y” should be interpreted as “X”, or “Y”, or “X and Y”.

All percentages and ratios are by weight unless otherwise indicated.

### Osteopontin (OPN)

Osteopontin (OPN) is a multifunctional protein present abundantly in human milk, but at low levels in cow's milk and infant formulas. According to Jiang et al. JPGN, 2020; 71; 125-131, human milk contains 170 to 220 mg OPN/L in colostrum, 120 to 140 mg OPN/L in transitional milk, and 60 to 80 mg/L in mid- and late lactation. Cow's milk and infant formulas, on the other hand, contain much lower concentrations of OPN; about 18 and 9 mg/L, respectively, cf. Schack et al. J Dairy Sci, 2009; 92; 5378-5385.

Importantly, the OPN receptor (integrin)-binding site is conserved across species, and it has been demonstrated that bovine OPN is bioactive in mice (Jiang et al. JPGN, 2020; 71; 125-131) and in humans (West et al. Pediatr Res, 2017; 82; 63-71).

Accordingly, in the present context the terms “osteopontin” or “OPN” refer to any mammalian milk OPN, including active truncated or cleaved forms thereof. The mammalian milk OPN may be obtained or derived from milk sources of human, cow (bovine), goat, sheep, camel buffalo, dromedary or llama origin. In a preferred embodiment of the invention, the OPN is bovine or human OPN, in particular bovine OPN.

Human OPN comprises 298 amino acids, whereas the bovine form only contains 262 residues, mainly because bovine OPN lacks a sequence of 22 residues corresponding to residues 188-209 in human OPN. Comparison of the bovine and human OPN sequences reveals that 182 amino acids are identical (61%) and an additional 44 residues retain high structural similarity. OPN is post-translationally modified by, in particular, phosphorylation, O-glycosylation, and proteolytic processing leading to the existence of several OPN isoforms. The amino acid sequences of human and bovine OPN are shown below (Christensen et al. Int Dairy J, 2016; 57; 1-6).

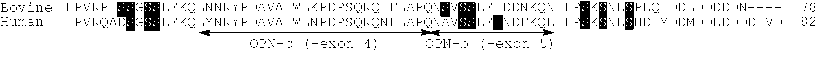

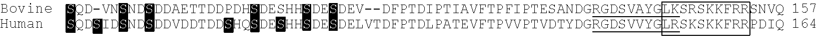

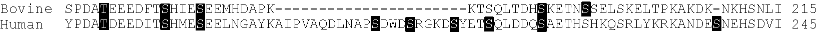

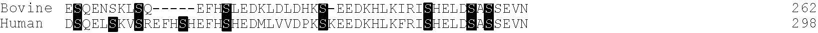

In the above figure, phosphorylation and glycosylation sites are highlighted in black and grey, respectively. The regions containing the identified cleavage sites in bovine and human OPN are boxed. Exons 4 and 5 missing in OPN-c and OPN-b are indicated. The integrin binding RGD and SVVYGLR-motifs are underlined and introduced gaps are indicated by dashed lines.

In both bovine and human milk, OPN is prone to proteolytic cleavage close to the RGD- and SVVYGLR sequences and hence a large fraction of milk OPN is found in a fragmented form with exposed integrin binding motifs (see the above figure). As will be understood, such cleaved and/or truncated forms of OPN are also encompassed by the terms “osteopontin” or “OPN”.

Whey protein concentrates enriched in bovine OPN (OPN-enriched WPC) are commercially available, such as Lactoprodan® OPN-10, which is sold by Arla Foods Ingredients, Viby, Denmark. Purified and lyophilised bovine OPN can also be obtained from Sigma. In a preferred embodiment of the invention, the OPN is included in the composition in the form of an OPN-enriched WPC. Mammalian OPN, in particular human or bovine OPN, may also be prepared by recombinant means as disclosed in Rollo et al, J Virol, 2005; 79; 3509-3516.

When the composition is in liquid form, the concentration of OPN is typically in the range from 25 to 250 mg/L, preferably from 50 to 200 mg/L, more preferably from 50 to 150 mg/L, even more preferably from 70 to 130 mg/L, most preferably from 75 to 125 mg/L. Specific examples of the concentration level of OPN, when the composition is in liquid form, include 50 to 75 mg/L, 75 to 100 mg/L, 100 to 200 mg/L or 100 to 150 mg/L.

When the composition is in solid form, the concentration of OPN is typically in the range from 0.015 to 0.15 wt % (g OPN per 100 g dry composition), preferably from 0.03 to 0.125 wt %, more preferably from 0.03 to 0.1 wt %, most preferably from 0.05 to 0.09 wt. Specific examples of the concentration level of OPN, when the composition is in dry form, include 0.03 to 0.05 wt % (g OPN per 100 g dry composition), 0.05 to 0.075 wt %, 0.075 to 0.125 wt % or 0.075 to 0.1 wt %.

### Composition

The composition to be used according to the invention is preferably a nutritional composition, in particular a nutritional composition selected from the group consisting of an infant formula, such as a starter infant formula or a follow-on formula; a baby food; an infant cereal composition; a growing-up milk; a fortifier, such as a human milk fortifier; and a supplement. The nutritional composition may be in either liquid or solid form, such as in powderous form. The nutritional composition typically comprises a protein component, a lipid (fat) component and a digestible carbohydrate component.

In the most preferred embodiment, the nutritional composition is an infant formula, such as a starter infant formula or a follow-on formula.

### Human Milk Oligosaccharides (HMOs)

In a highly preferred embodiment, the composition also comprises at least one human milk oligosaccharide (HMO).

In a further preferred embodiment, the composition comprises at least one HMO and at least one probiotic. In another preferred embodiment, the composition comprises at least one HMO and at least one non-digestible oligosaccharide, which is different from HMOs. In still another preferred embodiment, the composition comprises at least one non-digestible oligosaccharide, which is different from HMOs, and at least one probiotic. In yet another preferred embodiment, the composition comprises at least one HMO, at least one non-digestible oligosaccharide, which is different from HMOs, and at least one probiotic.

The at least one HMO may be selected from the group consisting of 2′-fucosyllactose (2FL), 3-fucosylactose (3FL), difucosyllactose (DFL), lacto-N-fucopentaose I (LNFP-I), lacto-N-fucopentaose II (LNFP-II), lacto-N-fucopentaose III (LNFP-III), lacto-N-fucopentaose V (LNFP-V), lacto-N-fucohexaose (LNFH), lacto-N-difucohexaose I (LDFH-I), lacto-N-tetraose (LNT), lacto-N-neotetraose (LNnT), lacto-N-hexaose (LNH), 3′-sialyllactose (3SL), 6′-sialyllactose (6SL), disialyllacto-N-tetraose (DSLNT), sialyllacto-N-tetraose (SLNT) and combinations thereof. In a preferred embodiment, the at least one HMO is selected from the group consisting of 2′-fucosyllactose (2FL), 3-fucosylactose (3FL), difucosyllactose (DFL), lacto-N-tetraose (LNT), lacto-N-neotetraose (LNnT), 3′-sialyllactose (3SL), 6′-sialyllactose (6SL) and combinations thereof. It is generally preferred that the composition comprises 2FL.

The composition may comprise a single HMO, e.g. a single HMO selected from the group consisting of 2FL, 3FL, DFL, LNFP-I, LNFP-II, LNFP-III, LNFP-V, LNFH, LDFH-I, LNT, LNnT, LNH, 3SL, 6SL, DSLNT and SLNT. Preferably, the single HMO is selected from the group consisting of 2FL, 3FL, DFL, LNT, LNnT, 3SL and 6SL. Most preferably, the single HMO is 2FL, i.e. 2FL is the only HMO present in the composition.

In another embodiment, the composition may comprise a mixture of two HMOs, e.g. two HMOs selected from the group consisting of 2FL, 3FL, DFL, LNFP-I, LNFP-II, LNFP-III, LNFP-V, LNFH, LDFH-I, LNT, LNnT, LNH, 3SL, 6SL, DSLNT and SLNT. Preferably, the two HMOs are selected from the group consisting of 2FL, 3FL, DFL, LNT, LNnT, 3SL and 6SL. It is generally preferred that one of the two HMOs is 2FL. As will be understood, it is also preferred that the two selected HMOs are the only HMOs present in the composition. In a particularly preferred embodiment, the composition comprises a mixture of 2FL and LNnT, i.e. 2FL and LNnT are the only HMOs present in the composition. In this case, the weight ratio between 2FL and LNnT is typically from 3:1 to 1:1, more preferably from 2.5:1 to 1.5:1, most preferably about 2:1.

In a further embodiment, the composition may comprise a mixture of three, four, five or six HMOs, e.g. three, four, five or six HMOs selected from the group consisting of 2FL, 3FL, DFL, LNFP-I, LNFP-II, LNFP-III, LNFP-V, LNFH, LDFH-I, LNT, LNnT, LNH, 3SL, 6SL, DSLNT and SLNT. Preferably, the three, four, five or six HMOs are selected from the group consisting of 2FL, 3FL, DFL, LNT, LNnT, 3SL and 6SL. It is generally preferred that one of the three, four, five or six HMOs is 2FL. As will be understood, it is also preferred that the three, four, five or six selected HMOs are the only HMOs present in the composition.

If the composition comprises a mixture of five HMOs, the five HMOs are preferably selected from the group consisting of 2FL, 3FL, DFL, LNFP-I, LNFP-II, LNFP-III, LNFP-V, LNFH, LDFH-I, LNT, LNnT, LNH, 3SL, 6SL, DSLNT and SLNT. More preferably, the five HMOs are selected from the group consisting of 2FL, 3FL, DFL, LNT, LNnT, 3SL and 6SL. It is generally preferred that one of the five HMOs is 2FL. As will be understood, it is also preferred that the five selected HMOs are the only HMOs present in the composition.

If the composition comprises a mixture of six HMOs, the six HMOs are preferably selected from the group consisting of 2FL, 3FL, DFL, LNFP-I, LNFP-II, LNFP-III, LNFP-V, LNFH, LDFH-I, LNT, LNnT, LNH, 3SL, 6SL, DSLNT and SLNT. More preferably, the six HMOs are selected from the group consisting of 2FL, 3FL, DFL, LNT, LNnT, 3SL and 6SL. It is generally preferred that one of the six HMOs is 2FL. As will be understood, it is also preferred that the six selected HMOs are the only HMOs present in the composition.

When the composition is in liquid form, the total HMO concentration is typically in the range from 0.5 to 10 g/L, preferably in the range from 1 to 7.5 g/L. Specific examples of the concentration level of total HMO, when the composition is in liquid form, include 1 to 5 g/L, 1 to 4 g/L, 2 to 5 g/L, 1 to 3 g/L or 2 to 4 g/L.

When the composition is in solid form, the total HMO concentration is typically in the range from 0.35 to 7 wt % (g total HMO/100 g dry composition), preferably in the range from 0.35 to 5 wt %. Specific examples of the concentration level of total HMO, when the composition is in dry form, include 0.5 to 3.5 wt % (g total HMO per 100 g dry composition), 0.5 to 2.5 wt %, 1 to 3.5 wt %, 0.5 to 2 wt % or 1 to 2.5 wt %.

Non-Digestible Oligosaccharides (Different from HMOs)

In a further interesting embodiment, the composition comprises at least one non-digestible oligosaccharide, which is different from HMOs. Accordingly, the composition may comprise fructooligosaccharides, galactooligosaccharides and/or galacturonic acid oligosaccharides. In a preferred embodiment, the composition comprises a mixture of galactooligosaccharides and fructooligosaccharides, in particular in a weight ratio of 9:1. Suitable non-digestible oligosaccharides are for example Vivinal GOS (FrieslandCampina DOMO), Raftilin HP or Raftilose (Orafti).

If the composition comprises non-digestible oligosaccharides, which are different from HMOs, these non-digestible oligosaccharides are typically present in an amount from 800 mg/L to 20 g/L, more preferably from 1.5 to 15 g/L, even more preferably from 3 to 10 g/L. Based on dry weight, the composition preferably comprises from 0.25 to 20 wt %, more preferably from 0.5 to 10 wt %, even more preferably 1.5 to 7.5 wt % non-digestible oligosaccharides, which are different from HMOs.

### Probiotics

In a still further interesting embodiment, the composition comprises at least one probiotic.

In a preferred embodiment of the invention, the probiotic is a Bifidobacterium or a Lactobacillus, or a mixture of at least one Bifidobacterium and at least one Lactobacillus.

When the probiotic is a Bifidobacterium, the Bifidobacterium is preferably selected from the group consisting of Bifidobacterium infantis, Bifidobacterium breve, Bifidobacterium longum, Bifidobacterium lactis, Bifidobacterium bifidum, Bifidobacterium adolescentis and Bifidobacterium animalis. In a more preferred embodiment, the Bifidobacterium is selected from the group consisting of Bifidobacterium infantis, Bifidobacterium breve, Bifidobacterium longum, Bifidobacterium lactis and Bifidobacterium bifidum. In an even more preferred embodiment, the Bifidobacterium is selected from the group consisting of Bifidobacterium infantis and Bifidobacterium lactis.

At the strain level, the Bifidobacterium is preferably selected from the group consisting of Bifidobacterium longum CNCM I-2170 (NCC 490), Bifidobacterium longum CNCM 1-2618 (NCC 2705), Bifidobacterium lactis CNCM I-3446 (NCC 2818), Bifidobacterium lactis BI07, Bifidobacterium lactis BL04, Bifidobacterium lactis HN019, Bifidobacterium infantis ATCC 17930 (LMG 11588) (NCC 3089) and Bifidobacterium infantis ATCC 15697 (NCC 3078), more preferably from the group consisting of Bifidobacterium infantis ATCC 17930 (LMG 11588) (NCC 3089), Bifidobacterium infantis ATCC 15697 (NCC 3078) and Bifidobacterium lactis CNCM I-3446 (NCC 2818).

When the probiotic is a Lactobacillus, the Lactobacillus is preferably selected from the group consisting of Lactobacillus acidophilus, Lactobacillus rhamnosus, Lactobacillus casei, Lactobacillus paracasei, Lactobacillus plantarum, Lactobacillus reuteri, Lactobacillus salivarius and Lactobacillus johnsonii.

At the strain level, the Lactobacillus is preferably selected from the group consisting of Lactobacillus johnsonii CNCM-I-1225 (NCC 533), Lactobacillus paracasei CNCM I-2116 (NCC 2461) and Lactobacillus rhamnosus GG ATCC 53103 (NCC 407).

Typically, the probiotic(s) will be present in the composition in an amount from 10³ to 10¹² cfu/g dry composition, preferably from 106 to 109 cfu/g dry composition.

### Proteins

The nutritional composition may comprise protein. The protein component typically provides 5 to 15% of the total calories. Preferably, the nutritional composition comprises a protein component that provides 6 to 12% of the total calories. More preferably, the protein component is present in the nutritional composition in an amount of at most 9% based on calories. More preferably the nutritional composition comprises from 7.2 to 8.0% protein based on total calories, in particular from 7.3 to 7.7% based on total calories. The protein concentration in the nutritional composition is determined by the sum of protein, peptides and free amino acids. Based on dry weight, the nutritional composition preferably comprises at most 12 wt % protein, more preferably of 9.6 to 12 wt %, even more preferably 10 to 11 wt %. Based on a ready-to-feed liquid product the nutritional composition preferably comprises at most 1.5 g protein/100 mL, more preferably of 1.2 to 1.5 g/100 mL, even more preferably of 1.25 to 1.35 g/100 mL.

The source of the protein should be selected in such a way that the minimum requirements for essential amino acid content are met and satisfactory growth is ensured. Hence, protein sources based on cow's milk proteins, such as whey, casein and mixtures thereof, and protein based on soy, potato or pea are preferred. In case whey proteins are used, the protein source is preferably based on acid whey, sweet whey, ideal whey, whey protein isolate or mixtures thereof and may include α-lactalbumin and β-lactoglobulin. More preferably, the protein source is based on acid whey or sweet whey from which caseino-glyco-macropeptide (CGMP) has been removed. Preferably, the composition comprises at least 3 wt % casein based on dry weight. The casein is preferably intact and/or non-hydrolysed. In the present context, “protein” also includes peptides and free amino acids. Accordingly, the protein component may also be hydrolysed protein, such as partially hydrolysed proteins, extensively hydrolysed proteins or amino acids.

### Lipid (Fat)

The nutritional composition may comprise lipid. The lipid component typically provides 30 to 60% of the total calories of the nutritional composition. More preferably, the nutritional composition comprises lipid providing 35 to 55% of the total calories, even more preferably the nutritional composition comprises lipid providing 40 to 50% of the total calories. When in liquid form, e.g. as a ready-to-feed liquid, the nutritional composition preferably comprises 2.1 to 6.5 g lipid/100 mL, more preferably 3.0 to 4.0 g/100 mL. Based on dry weight the nutritional composition preferably comprises 10 to 50 wt % lipid, more preferably 12.5 to 40 wt % lipid, even more preferably 19 to 30 wt % lipid.

Lipids include polar lipids (such as phospholipids, glycolipids, sphingomyelin, and cholesterol), monoglycerides, diglycerides, triglycerides and free fatty acids. Preferably, the nutritional composition comprises at least 75 wt %, more preferably at least 85 wt % triglycerides based on total lipids.

The lipid present in the nutritional composition typically comprises vegetable lipids. The presence of vegetable lipids advantageously enables an optimal fatty acid profile, high in (poly) unsaturated fatty acids and/or more reminiscent to human milk fat. Using lipids from cow's milk alone, or other domestic mammals, does not provide an optimal fatty acid profile. This less optimal fatty acid profile, such as a large amount of saturated fatty acids, is known to result in increased cholesterol levels. Preferably, the nutritional composition comprises at least one, preferably at least two lipid sources selected from the group consisting of linseed oil (flaxseed oil), rape seed oil (such as colza oil, low erucic acid rape seed oil and canola oil), sunflower oil, high oleic sunflower oil, safflower oil, high oleic safflower oil, olive oil, coconut oil, palm oil and palm kernel oil. More preferably, the nutritional composition comprises at least one, preferably at least two lipid sources selected from the group consisting of linseed oil, canola oil, coconut oil, sunflower oil and high oleic sunflower oil. Commercially available vegetable lipids are typically offered in the form a continuous oil phase.

When the nutritional composition is in liquid form, e.g. as a ready-to-feed liquid, the nutritional composition preferably comprises 2.1 to 6.5 g lipid/100 mL, more preferably 3.0 to 4.0 g/100 mL. Based on dry weight the nutritional composition preferably comprises 10 to 50 wt %, more preferably 12.5 to 40 wt % lipid, even more preferably 19 to 30 wt %. Preferably, the nutritional composition comprises 45 to 100 wt % vegetable lipids based on total lipids, more preferably 70 to 100 wt %, even more preferably 75 to 97 wt %. It is noted, therefore, that the nutritional composition also may comprise non-vegetable lipids. Suitable non-vegetable lipids include marine oils, microbial oils, egg fat and milk fat.

Triglycerides comprise a glyceride molecule to which, via ester bonds, three fatty acid residues are attached, which may be the same or different, and which are generally chosen from saturated and unsaturated fatty acids containing 6 to 26 carbon atoms, including, but not limited to LA, ALA, oleic acid (C18:1), palmitic acid (PA) (C16:0) and/or stearic acid (C18:0). Such fatty acid triglycerides may differ in the fatty acid residues that are present in the respective position(s) of the fatty acid residues (e.g. in the sn-1, sn-2 and/or sn-3 position).

In a particular preferred embodiment, the triglycerides used in the nutritional composition are chosen such that the amount of PA residues that are present in the triglycerides are 10 wt % or more based on total fatty acid present in the triglycerides, preferably more than 15 wt %. Preferably, the amount of PA residues that are present in the triglycerides are below 30 wt %, more preferably below 25%. Thus, the amount of PA residues that are present in the triglycerides may be in the range from 10 to 30 wt %, preferably from 15 to 25 wt %, in particular from 16 to 24 wt %, based on total fatty acid present in the triglycerides.

In addition, it is particularly preferred that the triglycerides used in the nutritional composition are chosen such that at least 30 wt %, preferably at least 35 wt %, more preferably at least 40 wt % of the PA residues are located in the sn-2 (or beta) position of the triglyceride.

Such triglycerides, with a high fraction of PA residues in the sn-2 position, are commercially available, e.g. from Loders Croklaan under the name Betapol® and/or can be prepared in a manner known per se, for instance as described in EP 0 698 078 and EP 0 758 846. Another suitable source is InFat® from Enzymotec. In case these lipids are obtained by trans- or interesterification of vegetable triglycerides, these sources are in the context of the present invention regarded as vegetable lipids.

Preferably the amount of triglycerides with a high fraction of PA residues in the sn-2 position that is comprised in the lipid fraction of the nutritional composition is typically between 10 and 100 wt %, preferably between 20 and 100 wt %, more preferably between 20 and 80 wt %, even more preferably between 50 and 80 wt %.

Another preferred source of triglycerides with a high fraction of PA residues in the sn-2 position is non-human animal fat, more preferably non-human mammalian milk fat, even more preferably cow's milk fat. Preferably, non-human mammalian milk fat, in particular cow's milk fat, is used in the form of anhydrous milk fat or butter oil. Preferably the amount of milk fat is between 10 and 100 wt % based on total lipid, preferably between 10 and 80 wt % based on total lipid, more preferably between 20 and 80 wt %, even more preferably between 20 and 50 wt %, most preferably between 25 and 50 wt. % based on total lipid.

In a preferred embodiment, the nutritional composition comprises phospholipids. Phosholipids are polar lipids, which are amphipathic of nature and include glycerophospholipids, glycosphingolipids, sphingomyelin and/or cholesterol. The phospholipids are typically present in an amount corresponding to 0.25 to 10 wt % phospholipids based on total lipid, more preferably 0.5 to 10 wt %, more preferably 1 to 10 wt %, even more preferably 2 to 10 wt % most preferably 3 to 8 wt % phospholipids based on total lipid.

Linoleic acid (LA) is present in a sufficient amount in order to promote a healthy growth and development, yet in an amount as low as possible to prevent occurrence of obesity later in life. The composition therefore preferably comprises less than 15 wt % LA based on total fatty acids, preferably from 5 to 14.5 wt %, more preferably of 6 to 10 wt %.

Preferably, α-linolenic acid (ALA) is present in a sufficient amount to promote a healthy growth and development. The nutritional composition therefore preferably comprises at least 1.0 wt % ALA based on total fatty acids. Preferably the nutritional composition comprises at least 1.5 wt % ALA based on total fatty acids, more preferably at least 2.0 wt %. Typically, the nutritional composition comprises less than 10 wt % ALA, more preferably less than 5.0 wt % ALA, based on total fatty acids. The weight ratio LA/ALA should be well-balanced in order to prevent obesity and hypertriglyceridaemia later in life, while at the same time ensuring a normal growth and development. Therefore, the nutritional composition preferably comprises LA and ALA in a weight ratio (LA/ALA) of 2 to 15, more preferably of 2 to 7, even more preferably of 4 to 7, still more preferably of 3 to 6, most preferably of 4 to 5.5, in particular of 4 to 5.

Preferably, the nutritional composition comprises n-3 LC-PUFA since n-3 LC-PUFA have a beneficial effect on cardiovascular disease risk and vascular fatty acid membrane composition. More preferably, the present composition comprises EPA, DPA and/or DHA, in particular DHA. Since a low concentration of DHA, DPA and/or EPA is already effective and normal growth and development are important, the content of n-3 LC-PUFA in the nutritional composition does typically not exceed 15 wt % of the total fatty acid content, preferably it does not exceed 10 wt %, even more preferably it does not exceed 5 wt %. Preferably, the nutritional composition comprises at least 0.2 wt % n-3 LC-PUFA based on the total fatty acid content, preferably at least 0.5 wt %, more preferably at least 0.75 wt %. Preferably, the nutritional composition comprises at least 0.2 wt %, preferably at least 0.5 wt %, more preferably at least 0.75 wt % of the sum of DHA, DPA and EPA based on total fatty acid content. Preferably, the sum of DHA, DPA and EPA does not exceed 5 wt % of the total fatty acid content.

Preferably, the nutritional composition also comprises n-6 LC-PUFA since n-6 LC-PUFA, in particular ARA, is important for infants for optimal functional membranes, especially membranes of neurological tissues. The n-6 LC-PUFA content preferably does not exceed 5 wt %, more preferably, it does not exceed 2.0 wt %, more preferably it does not exceed 0.75 wt %, even more preferably it does not exceed 0.5 wt %, based on total fatty acids, the amount of n-6 LC-PUFA is preferably at least 0.02 wt %, more preferably at least 0.05 wt %, more preferably at least 0.1 wt % based on total fatty acids. The presence of ARA is advantageous in an infant formula that is low in LA since it remedies LA deficiency. The presence of, preferably low amounts, of ARA is therefore beneficial in nutrition to be administered to infants below the age of 6 months, since for these infants the infant formula is generally the only source of nutrition.

Lipid from fish oil (preferably tuna fish oil) and single cell oil (such as algal, microbial oil and fungal oil) are suitable LC-PUFA sources. Preferably as a source of n-3 LC-PUFA single cell oil, including algal oil and microbial oil, is used, since these oil sources have a low EPA/DHA ratio. Thus, in a further embodiment, the nutritional composition further comprises at least one lipid selected from the group consisting of fish oil, marine oil, algal oil, fungal oil and microbial oil.

### Digestible Carbohydrates

The nutritional composition may comprise digestible carbohydrate. The digestible carbohydrate typically provides 30 to 80% of the total calories of the composition. Preferably, the digestible carbohydrate provides 40 to 60% of the total calories. When in liquid form, e.g. as a ready-to-feed liquid, the nutritional composition preferably comprises 3.0 to 30 g digestible carbohydrate/100 mL, more preferably 6.0 to 20 g/100 mL, even more preferably 7.0 to 10.0 g/100 mL. Based on dry weight the nutritional composition preferably comprises 20 to 80 wt %, more preferably 40 to 65 wt % digestible carbohydrates.

Preferred digestible carbohydrate sources are lactose, glucose, sucrose, fructose, galactose, maltose, starch and maltodextrin. Lactose is the main digestible carbohydrate present in human milk, and the nutritional composition preferably comprises lactose. The nutritional composition typically comprises digestible carbohydrate, wherein at least 35 wt %, preferably at least 50 wt %, more preferably at least 75 wt %, even more preferably at least 90 wt %, most preferably at least 95 wt % of the digestible carbohydrate is lactose. Based on dry weight the nutritional composition preferably comprises 20 to 80 wt %, more preferably 40 to 65 wt % lactose.

### Miscellaneous

The nutritional composition is particularly suitable for providing the daily nutritional requirements to a human with an age below 36 months, particularly an infant with the age below 24 months, even more preferably an infant with the age below 18 months, most preferably below 12 months of age. The nutritional composition is not human breast milk. The nutritional composition is not (raw) cow's milk or other (raw) mammalian milks. The nutritional composition preferably comprises other ingredients, such as vitamins and minerals according to international directives for infant formulas.

In order to meet the caloric requirements of the infant, the nutritional composition preferably comprises from 50 to 100 kcal/100 mL liquid, more preferably from 60 to 90 kcal/100 mL liquid, even more preferably from 60 to 75 kcal/100 mL liquid, most preferably from 60 to 70 kcal/100 ml liquid. This caloric density ensures an optimal ratio between water and calorie consumption.

The nutritional composition may be in a liquid form with a viscosity below 35 mPa·s, more preferably below 6 mPa·s as measured in a Brookfield viscometer at 20° C. at a shear rate of 100 s⁻¹. In one embodiment, the nutritional composition is a powder. Suitably, the composition is in a powdered form, which can be reconstituted with water or other food grade aqueous liquids, to form a liquid, or in a liquid concentrate form that should be diluted with water. When the nutritional composition is in a liquid form, the preferred volume administered on a daily basis is in the range from about 80 to 2500 mL, more preferably from about 450 to 1000 mL per day.

### Therapeutic Applications

The composition is effective for use in therapy.

The composition is, in particular effective for use in the treatment and/or prevention of a viral infection in a mammal, such as a human, in particular an infant or a young child.

Accordingly, the composition is also effective for use in reducing the risk of contracting a viral infection in a mammal and/or for use in reducing the symptoms associated with a viral infection in a mammal, such as a human, in particular an infant or a young child.

In one embodiment of the present invention, the symptoms associated with a viral infection are symptoms which are caused by the viral infection (i.e. not simply occurring at the same time).

The viral infection may be a viral gastrointestinal infection or a viral respiratory tract infection, including a viral upper respiratory tract infection or a viral lower respiratory tract infection.

In a preferred embodiment, the viral infection is a viral respiratory tract infection. The viral respiratory tract infection may be a viral infection in the upper respiratory tract or in the lower respiratory tract. In a typical embodiment of the invention, the viral respiratory tract infection is caused by RSV.

The disease associated with the viral infection will be typically be common cold, influenza (flu), bronchitis, bronchiolitis, pneumonia, sore throat (pharyngitis), sinusitis, non-allergic rhinitis, severe acute respiratory syndrome (SARS), viral croup, otitis media, meningitis or diarrhoea. Typically, when the infection is in the viral respiratory tract, the disease associated with the respiratory tract infection is common cold, influenza (flu), bronchitis, bronchiolitis, pneumonia, sore throat (pharyngitis), sinusitis, non-allergic rhinitis, severe acute respiratory syndrome (SARS), viral croup or otitis media. More often, the disease associated with the viral respiratory tract infection is common cold, influenza (flu), bronchitis, bronchiolitis or pneumonia, in particular bronchiolitis or pneumonia.

Accordingly, in a preferred embodiment of the invention the composition is for use in treating and/or preventing a disease associated with a viral respiratory tract infection selected from the group consisting of common cold, influenza (flu), bronchitis, bronchiolitis and pneumonia. In a more preferred embodiment, the disease associated with the respiratory tract infection is selected from the group consisting of bronchiolitis and pneumonia, in particular bronchiolitis and pneumonia caused by RSV. In an even more preferred embodiment, the disease associated with the respiratory tract infection is bronchiolitis, in particular bronchiolitis caused by RSV.

The symptoms most often associated with the viral infection, and which may be reduced by the composition, are irritation in the lungs, congestion in the lungs, excessive mucus production, fever, cough, wheezing, breathlessness, abdominal cramps, diarrhoea or vomiting.

The above-mentioned infections may be caused by a variety of different viruses, including respiratory syncytial virus (RSV), parainfluenza virus (PIV), influenza virus such as influenza virus A (IVA) and/or influenza virus B (IVB), rhinovirus (RV), adenovirus (ADV), metapneumovirus (MPV), bocavirus (BoV), coronavirus (CoV), myxovirus, herpesvirus, enterovirus (EV), parachovirus (PeV), flavivirus including zikavirus (ZIKV) and/or West Nile virus (WNV), or a combination thereof. In one embodiment, the above-mentioned infections may be caused by a variety of different viruses, including respiratory syncytial virus (RSV), parainfluenza virus (PIV), influenza virus such as influenza virus A (IVA) and/or influenza virus B (IVB), rhinovirus (RV), adenovirus (ADV), metapneumovirus (MPV), bocavirus (BoV), coronavirus (CoV), myxovirus, herpesvirus, enterovirus (EV), parachovirus (PeV), or a combination thereof.

In on embodiment of the present invention, above-mentioned infections may be caused by a variety of different viruses which are capable of mutating from one person to the next so that vaccination for these kinds of viruses is either not possible or very difficult, as the viruses have already changed their format by the time vaccines are developed. In such embodiment, above-mentioned infections may be caused by a variety of different viruses selected in the group consisting of: respiratory syncytial virus (RSV), parainfluenza virus (PIV), influenza virus such as influenza virus A (IVA) and/or influenza virus B (IVB), rhinovirus (RV), adenovirus (ADV), metapneumovirus (MPV), bocavirus (BoV), coronavirus (CoV), myxovirus, herpesvirus, enterovirus (EV), parachovirus (PeV), and a combination thereof.

The composition is particularly effective in treating, preventing, reducing the risk of contracting and/or reducing the symptoms of a viral infections caused by respiratory syncytial virus (RSV). Thus, the composition is particularly preferred for use in treating, preventing, reducing the risk of contracting and/or reducing the symptoms of bronchiolitis caused by respiratory syncytial virus (RSV) or pneumonia caused by respiratory syncytial virus (RSV).

Respiratory viral infections are also a major determinant for development of pulmonary diseases, such as chronic obstructive pulmonary disease (Savran et al, Int J Chron Obstruct, 2015; 191; 34-44), later in life. Thus, the composition is also suitable for use in preventing or reducing the risk of developing pulmonary diseases, in particular chronic obstructive pulmonary disease, in a mammal, such as a human. In particular, the composition is also suitable for use in preventing or reducing the risk of developing pulmonary diseases, in particular chronic obstructive pulmonary disease, in a mammal, such as a human, later in life.

Since viral infections, in particular infection with RSV, are often associated with bacterial co-infection (Thorburn et al, Thorax, 2006; 61 (7); 611-615) or secondary infection (Sande et al, Nature Communications, 2019; 10; 2218), including antibiotic use, the composition is also effective for use in preventing or reducing the risk of a bacterial co-infection and/or a bacterial secondary infection associated with respiratory viral infection in a mammal, in particular a human. Pathogenic bacteria typically involved in co-infections or secondary infections include Staphylococcus aureus, Streptococcus pneumoniae and/or Haemophilus influenza. In an interesting embodiment of the invention, the bacterial co-infection and/or a bacterial secondary infection is associated with antibiotic use.

The mammal to be treated is preferably a human being, but the mammal be also be non-human mammal, such as a non-human mammal selected from the group consisting of pig, cow, horse, dog, cat, goat, sheep and rabbit.

The composition is useful for treating and/or preventing infections, in particular respiratory tract infection in a human of any age. Thus, the human to be treated with the composition may have an age selected from the group consisting of 0 to <1 year (infants), 1 to <3 years (young children), 3 to <5 years (pre-schoolers), 5 to <13 years (grade-schoolers), 13 to <20 years (teenagers), 20 years or older (adults), 20 to <40 years (young adults), 40 to <60 years (middle-aged adults), 60 to <75 years (seniors) and 75 years or older (elderly).

The composition is particularly useful for being fed to a human with an age below 36 months, particularly an infant with the age below 24 months, even more preferably an infant with the age below 18 months, most preferably below 12 months of age. In a particular preferred embodiment, the human is selected from the group consisting of infants and young children, in particular an infant, e.g. an infant having an age from 0 to 6 months.

In general, formula-fed infants have an underdeveloped immune system compared with adults and are more prone to viral infections than breastfed, and the younger the infant is, the less developed the immune system. Accordingly, the composition is particularly useful for preterm infants and/or low or very low birth weight infants, since these infants are even more vulnerable and prone to viral infections. In another particularly interesting embodiment, the composition is used in infants delivered via Caesarean section. Caesarean section born infants are born in a hospital in an environment having more pathogens against which the antibodies, transferred from the mother to the infant, are not effective against. Caesarean section born infants have a delayed and less optimal colonization of the large intestinal tract and are therefore also more prone to infections.

Since viral infections, in particular infection with RSV, is associated with subsequent development of allergic airway diseases, such as asthma, later in life (Feldman et al, Am J Respir Crit Care Med, 2015; 191; 34-44), the composition is also effective for use in preventing or reducing the risk of allergen sensitisation and/or developing an allergic respiratory tract disease in a mammal, such as a human. Examples of allergic respiratory tract diseases include asthma and recurrent wheeze, in particular asthma.

In this case, the composition is preferably administered to a human having an age from 0 to <3 years, preferably from 0 to 2 years, more preferably from 0 to <1 year, such as from 0 to 6 months. This, in turn, prevents or reduces the risk of developing an allergic respiratory tract disease later in life, i.e. when the human is no longer being administered the composition, e.g. when said human has reached an age of 3 years or more.

In an embodiment of the invention, the composition is not for use in enhancing the immune resistance to an infectious disease in a mammal when the immune resistance is induced by vaccination.

### Examples

### Materials and Methods

### The Neonatal Mouse Model of Pneumonia Virus of Mice (PVM)-Induced Bronchiolitis

C57BL/6 pups were either nursed by OPN-sufficient or OPN-deficient dams.

At postnatal day 5 (PND5), mice receiving OPN-deficient mother's milk were fed with either a whey protein concentrate (WPC) enriched in OPN (OPN_{enriched} group) or a control WPC (OPN− group) once daily for 15 consecutive days.

In a similar way, at PND5, mice receiving OPN-sufficient mother's milk were fed with control WPC (OPN+ group) once daily for 15 consecutive days.

At PND10, all animals were infected intra-nasally (i.n.) with 10 PFU of PVM (J3666) to induce bronchiolitis.

The used OPN-enriched WPC contained 79 g protein per 100 g, and 729 mg OPN per 100 g. The pups in the OPN_{enriched} group were fed the OPN-enriched WPC as a supplement to deliver 12 μg OPN per average gram of bodyweight per day.

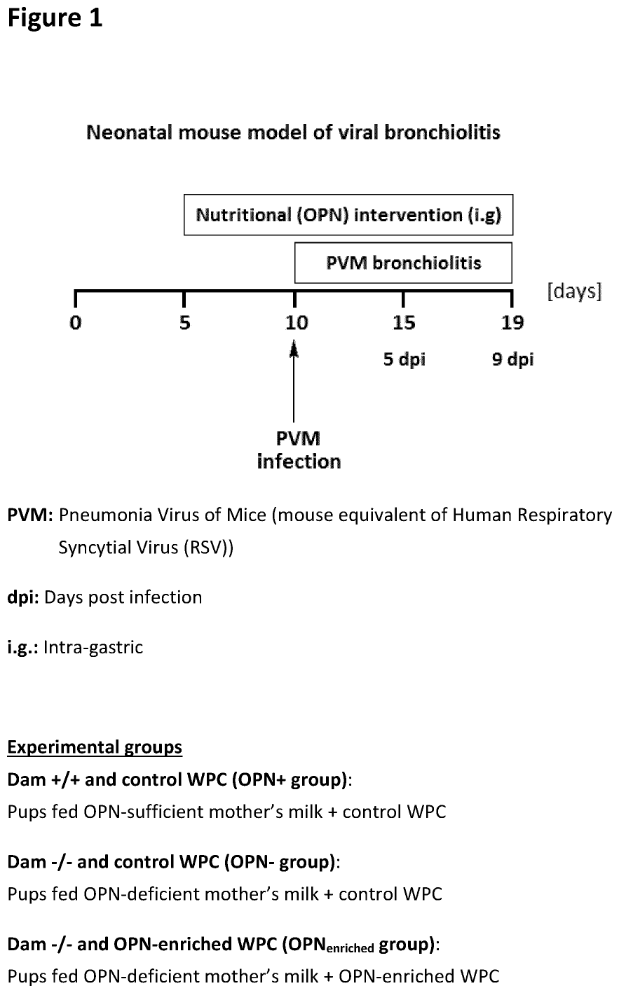

The study is outlined in FIG. 1.

### Results

### Example 1

Intervention with OPN in Early Life Reduces Viral Burden in the Airways

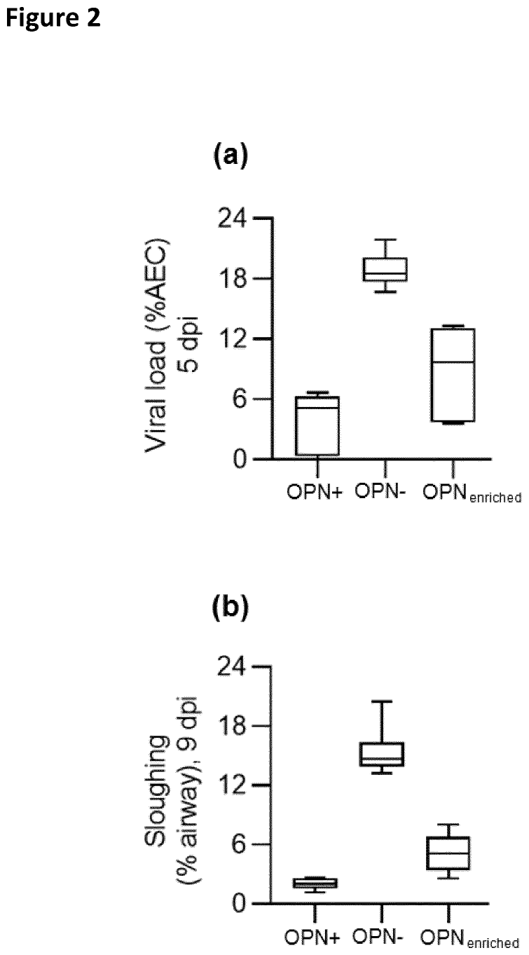

To assess whether OPN intervention has an impact on pathogen expansion, viral load in PVM-infected mice was assessed as the percentage of PVM-positive airway epithelial cells (AEC) stained with an antibody against a specific PVM-glycoprotein. Surprisingly, OPN intervention resulted in a significant decrease in virus infected AECs 5 days post infection (dpi) in the OPN_{enriched} group compared to the OPN− group (FIG. 2a). AEC detachment is a key feature of viral bronchiolitis and known to be associated with disease severity and viral load. Sloughing of the airway epithelium was quantified by measuring the length of sloughed airway epithelium and expressing this as a percentage of the basement membrane length of a single airway. AEC sloughing was significantly elevated in the OPN− group at 9 dpi, whereas sloughing in the OPN_{enriched} group was close to the OPN+ group at 9 dpi (FIG. 2b).

These results demonstrate that OPN intervention in early life lowers the viral burden and severity of infection.

### Example 2

Intervention with OPN in Early Life Promotes an Appropriate Immune Response

Plasmacytoid dendritic cells (pDCs) are critical regulators of viral infections. pDCs recognize invading viruses and rapidly produce vast amounts of type 1 and type 3 interferons. This process promotes an antiviral state and the airway epithelium fortifies itself against the virus using different mechanisms. An appropriate immune response is generated, the virus gets cleared and pDCs then contribute to immune regulation to dampen local inflammation and restore tissue homeostasis.

To investigate whether OPN intervention has an impact on immune cell composition, the number of pDCs, CD4+ and CD8+ T cells and neutrophils in lung tissues 5 and/or 9 days post infection were enumerated using multi-colour flow cytometry. Isolated single-cell suspensions from the lungs were washed with PBS/2% FCS, and red blood cells were lysed using Gey's buffer. Cells were washed again, followed by incubation with anti-FcγRIII/II for 15 min at 4° C. Cells were stained using fluorescently labeled antibodies described in the gating strategies herein, with DAPI, 7-AAD, or Zombie NIR used to exclude dead cells.

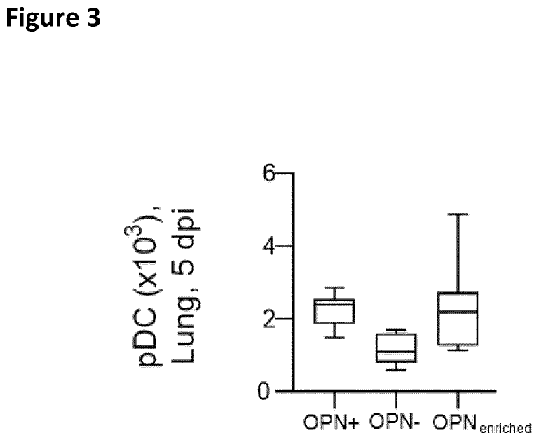

The OPN_{enriched} group showed an increased number of pDCs (defined as live, CD45RA⁺, CD11c⁺, CD11b⁻ B220⁺, Siglec-H⁺ cells) in the lung compared to the OPN− group (FIG. 3).

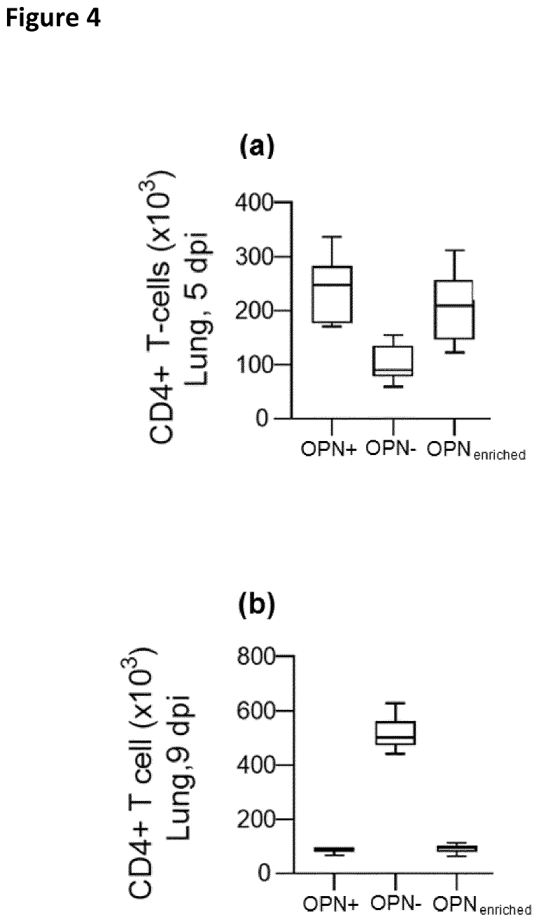

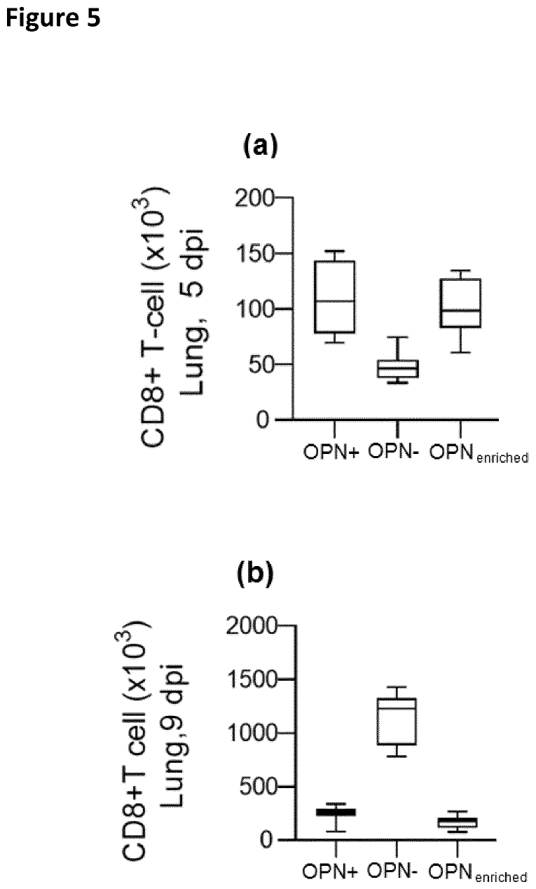

The kinetics of the number of CD4⁺ and CD8+ T cells in lung tissues of PVM-infected mice serves as a measure of the immune defence mounted against the viral infection and appropriate resolution of the immune response upon viral clearance. At 5 days post infection (peak of viral infection), OPN intervention resulted in a significantly higher number of CD4⁺ cells (defined as live, CD45⁺, CD3ε⁺, CD4⁺) in lung tissues in the OPN_{enriched} group as compared to the OPN− group (FIG. 4a). Importantly, CD4⁺ T cell accumulation in the lungs in the OPN_{enriched} group was tightly regulated with a significant decline over the OPN− group at 9 days post infection (peak of immunopathology), indicating a rapid resolution of lung tissue inflammation (FIG. 4b). A similar kinetic profile as for CD4⁺ cells was observed with CD8⁺ cell numbers (defined as live, CD45⁺, CD3ε⁺, CD8⁺) in infected lungs (FIGS. 5a and 5b).

### Example 3

Intervention with OPN in Early Life Reduces Inflammatory Neutrophil Infiltration to the Airways and Attenuates Pathological Airway Smooth Muscle Remodeling

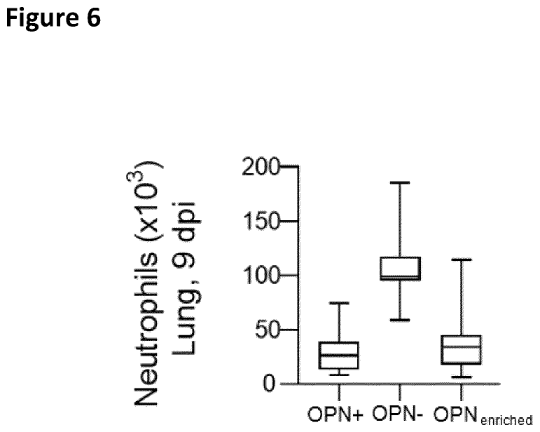

Granulocytes, such as eosinophils and neutrophils, infiltrate the airways in response to PVM infection and serve as reliable biomarkers for bronchiolitis severity. Intervention with OPN was associated with reduced granulocytic inflammation as evidenced by neutrophil infiltration (defined as live, CD45⁺, lineage, Gr-1⁺, CD11b⁺, Ly6G⁺ cells) to the airways (9 dpi) (FIG. 6).

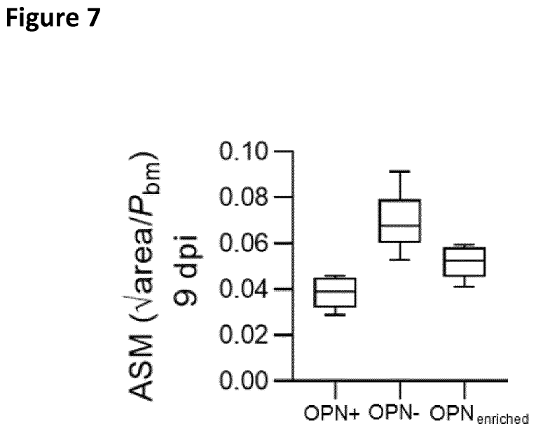

In line with the reduced neutrophil infiltration in response to PVM infection (9 dpi), OPN intervention in early life attenuated pathological airway smooth muscle remodeling in the OPN_{enriched} group as assessed by immunohistochemical staining of α-smooth muscle actin, while airway smooth muscle remodeling was dysregulated in the OPN− group (FIG. 7).

In summary, these results deliver strong evidence that dietary intervention with OPN in early life provides protection from viral bronchiolitis, supports rapid resolution of lung inflammation and attenuates pathological airway smooth muscle remodeling known to increase the risk of chronic inflammatory diseases such as asthma or COPD later in life.

1. A method for the treatment and/or prevention of a viral infection in a mammal comprising administering a composition comprising osteopontin (OPN) to the mammal.

2. The method according to claim 1, wherein said viral infection is a viral respiratory infection.

3. The method according to claim 1, wherein said viral infection causes a disease selected from the group consisting of bronchitis, bronchiolitis and pneumonia.

4. (canceled)

5. The method according to claim 1, wherein the mammal has symptoms associated with the viral infection selected from the group consisting of irritation in the lungs, congestion in the lungs, excessive mucus production, fever, cough, wheezing, breathlessness, abdominal cramps, diarrhoea and vomiting.

6. The method according to claim 5, wherein symptoms associated with a viral infection are symptoms which are caused by the viral infection.

7. The method according to claim 1, wherein said infection is caused by respiratory syncytial virus (RSV).

8. The method according to claim 1, wherein said infection is caused by respiratory syncytial virus (RSV) and wherein said disease is bronchiolitis.

9. The method according to claim 1, wherein said mammal is a human.

10. The method according to claim 9, wherein said human is an infant or a young child (toddler).

11. The method according to claim 1, wherein said composition comprises at least one non-digestible oligosaccharide, which is different from human milk oligosaccharides (HMOs).

12. The method according to claim 1, wherein said composition comprises at least one human milk oligosaccharide (HMO).

13. The method according to claim 12, wherein said at least one HMO is selected from the group consisting of 2′-fucosyllactose (2FL), 3-fucosylactose (3FL), difucosyllactose (DFL), lacto-N-fucopentaose I (LNFP-I), lacto-N-fucopentaose II (LNFP-II), lacto-N-fucopentaose III (LNFP-III), lacto-N-fucopentaose V (LNFP-V), lacto-N-fucohexaose (LNFH), lacto-N-difucohexaose I (LDFH-I), lacto-N-tetraose (LNT), lacto-N-neotetraose (LNnT), lacto-N-hexaose (LNH), 3′-sialyllactose (3SL), 6′-sialyllactose (6SL), disialyllacto-N-tetraose (DSLNT), sialyllacto-N-tetraose (SLNT) and combinations thereof.

14. (canceled)

15. The method according to claim 1, wherein said composition comprises at least one probiotic.

16. The method according to claim 1, wherein said composition is a nutritional composition.

17. The method according to claim 16, wherein said nutritional composition is selected from the group consisting of an infant formula, such as a starter infant formula or a follow-on formula; a baby food; an infant cereal composition; a growing-up milk; a fortifier, such as a human milk fortifier; and a supplement.

18. (canceled)

19. The method according to claim 1, wherein the viral infection is caused by a virus selected in the group consisting of respiratory syncytial virus (RSV), parainfluenza virus (PIV), influenza virus such as influenza virus A (IVA) and/or influenza virus B (IVB), rhinovirus (RV), adenovirus (ADV), metapneumovirus (MPV), bocavirus (BoV), coronavirus (CoV), myxovirus, herpesvirus, enterovirus (EV), parachovirus (PeV), and a combination thereof.

20. (canceled)

21. A method for use in preventing or reducing the risk of allergen sensitisation and/or developing an allergic respiratory tract disease in a mammal comprising administering osteopontin (OPN) to the mammal.

22. A method for use in preventing or reducing the risk of pulmonary diseases, including chronic obstructive pulmonary disease, in a mammal comprising administering osteopontin (OPN) to the mammal.

23. (canceled)


SEQUENCE LISTING (2 sequences)

SEQ ID NO: 1 - protein, 262 aa, Bos taurus
LPVKPTSSGSSEEKQLNNKYPDAVATWLKPDPSQKQTFLAPQNSVSSEETDDNKQNTLPSKSNESPEQTDDLDDDDDNSQDVNSNDSDDAETTDDPDHSDESHHSDESDEVDFPTDIPTIAVFTPFIPTESANDGRGDSVAYGLKSRSKKFRRSNVQSPDATEEDFTSHIESEEMHDAPKKTSQLTDHSKETNSSELSKELTPKAKDKNKHSNLIESQENSKLSQEFHSLEDKLDLDHKSEEDKHLKIRISHELDSASSEVN

SEQ ID NO: 2 - protein, 298 aa, Homo sapiens
IPVKQADSGSSEEKQLYNKYPDAVATWLNPDPSQKQNLLAPQNAVSSEETNDFKQETLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVN